(19) United States
(12) Patent Application Publication
(10) Pub. No.: US 2005/0118203 A1
(43) Pub. Date: Jun. 2, 2005

(54) Mixing powder of plant sterol and emulsifier, and method for preparing the same

(76) Inventors: Won-Tae Yoon, Seoul (KR); Kab-Sig Kim, Koyang (KR); Tae-Jin Kim, Seoul (KR); Hyung-Pyo Hong, Buchon (KR)

Correspondence Address: Drinker Biddle & Reath, One Logan Square, 18th & Cherry Streets, Philadelphia, PA, 19103-6996, US

(21) Appl. No.: 10/508,690

(22) PCT Filed: Mar. 20, 2002
(86) PCT No.: PCT/KR02/00473

(51) Int. Cl. A61K 31/56; A61K 9/00

(52) U.S. Cl. 424/400; 514/169

(57) ABSTRACT

Disclosed are a mixing powder of plant sterol and emulsifier, and method for preparing the same. More particularly, the present invention provides a mixing powder of plant sterol and emulsifier for application to almost all food, irrespective of food base, to obtain an improvement in the dispersion stability of the plant sterol micelles inhibiting the absorption of cholesterol.

### TECHNICAL FIELD

The present invention relates to a powdered mixture of plant sterol and an emulsifier and a method for preparing the same. More particularly, the present invention relates to a powdered mixture of plant sterol and emulsifier, which is such a size that it is greatly improved in bio-availability, leading to a decrease in serum cholesterol level even with little ingestion, and which can be applied to various foods irrespective of food bases, with excellent dispersion stability.

### BACKGROUND ART

Found in large concentrations in the brain, nervous tissues, organs, and blood plasma of higher animals, cholesterol, a kind of steroids, is the major precursor of the synthesis of vitamin D and various steroid hormones, including sex hormones (testosterone, progesterone, etc.,), adrenal cortical hormone, bile acid, etc. High levels of cholesterol in the blood are associated with an increased risk of cardiovascular diseases, such as hyperlipidemia, arteriosclerosis, arrhythmia, cardiac infarction, and so on. As a result of over-ingestion of cholesterol, diseases associated with cholesterol are becoming an increasingly big social problem.

It is known that both endogenic and dietary cholesterol move into the small intestine and about 50% thereof is absorbed from the intestines (Bosner, M. S., Ostlund, R. E., Jr., Osofisan, O., Grosklos, J., Fritschle, C., Lange, L. G. 1993). Based on this fact, a mechanism for preventing cholesterol from being absorbed from intestines is of special interest to those who have made efforts to discover clues for the prophylaxis and treatment of cholesterol-associated diseases.

Naturally occurring in a broad spectrum of plants such as bean, corn, wood, tallow oil, etc., plant sterol (or phytosterol) or plant stanol (or phytostanol) is non-toxic. Plant sterol or phytosterol can be broken down into sitosterol, campesterol, and stigmasterol, while plant stanol or phytostanol comprises sitostanol and campestanol. For purposes of convenience, they are all called plant sterol herein.

With structures very similar to that of cholesterol, plant sterol, when ingested in large quantities, is known to inhibit the absorption of intestinal and bile cholesterol, thereby reducing the serum cholesterol level, as disclosed in U.S. Pat. No. 5,578,334. By taking advantage of the inhibitory function of plant sterol against cholesterol absorption, clinical trials have been conducted of plant sterol as a therapeutic agent for treatment of cardiovascular diseases, coronary artery diseases and hyperlipidemia (Atherosclerosis 28:325-338).

Despite this useful function, plant sterol is difficult to apply to foods on account of its physical properties, that is, very poor solubility in both water and oil. Accordingly, there have been developed foods with only limited content of plant sterol.

In order to increase the solubility of plant sterol, some researchers have synthesized various derivatives of plant sterol. For example, sitosterol mixed in certain ratios with starch hydrolysate, silicon dioxide and polyoxylene sorbitan monostearate through homogenizing, deaeration, pasteurizing and evaporation steps to form a medicinal powder for oral application, as disclosed in U.S. Pat. No. 3,881,005. U.S. Pat. No. 5,932,562 discloses an aqueous homogeneous micellar mix of a plant sterol, lecithin and lysolecithin which has been dried to a finely divided water soluble powder. This was obtained by mixing plant sterol, lecithin and lysolecithin together in chloroform at a fixed molar ratio and removing the chloroform therefrom.

Other water-soluble plant sterols can be found in U. S. Pat. Nos. 6,054,144 and 6,110,502. According to these patents, aqueous-dispersible plant sterol is produced by admixing oryzanol or plant sterol, a monofunctional surfactant and polyfunctional surfactant in water at fixed ratios, and drying the admixture. This production method is characterized by being free from homogenization and deaeration steps with adoption of polyoxylene sorbitan monopalmitate and sorbitan monopalmitate as a monofunctional surfactant and a polyfunctional surfactant, respectively.

U.S. Pat. No. 6,190,720 discloses a food ingredient that can be used as a cholesterol-lowering agent, teaching that the food ingredient can be prepared by combining one or more molten plant sterols with one or more fats and one or more emulsifiers to homogeneity and cooling the homogeneous mixture to about 60° C. under agitation to give a paste. This food ingredient can be applied to oil-based foods such as salad dressings, margarine, etc.

Cholesterol reducing, edible products can be found in PCT WO 00/33669. According to the method of this patent, plant sterols are dissolved or mixed in a melt of a food emulsifier, admixed with protein-containing foods such as milk or yogurt, homogenized, and added to food products. The dispersion stability of the cholesterol reducing, edible products is maintained only in the presence of a protein-containing material.

U.S. Pat. No. 6,267,963 is concerned with a plant sterol-emulsifier complex which has a melting temperature at least 30° C. below that of the plant sterol, characterized in that, due to its reduced melting temperature, the plant sterol-emulsifier is less likely to crystallize during or after the manufacture of food products, and can be incorporated into food products in an amount effective to reduce serum cholesterol levels in a human consuming the food products without unpleasant effects on the texture of the food products.

As described above, the preparation of powdered mixtures of plant sterol and emulsifiers has resorted to a sequence of processes including homogenization, deaeration, sterilization, and evaporization, or utilized organic solvents such as chloroform which are removed following the dissolution of the ingredients therein. Alternatively, a large quantity of emulsifiers are used to prepare powdered mixtures for use in foods. However, the powdered mixtures prepared by such conventional methods are poor in terms of dispersion stability in water, in addition to being applied to a limited range of food bases, particularly when stable dispersion in water is required.

### DISCLOSURE OF THE INVENTION

Therefore, it is an object of the present invention is to provide a powdered mixture of plant sterol and an emulsifier, in which the plant sterol and the emulsifier are homogeneously dispersed in such a fine size level that it is improved in bio-availability and can be applied to various foods, irrespective of food bases, with no influence on the characteristic taste and flavor of the applied food, and without providing a bristly sensation in the mouth.

It is another object of the present invention to provide a method for preparing the powdered mixture of plant sterol and an emulsifier, which is applied to foods to inhibit the absorption of intestinal cholesterol and bile cholesterol even when it is ingested in a relatively small amount thanks to the high bio-availability of the plant sterol contained therein, in addition to not producing a bristly sensation in the mouth.

In accordance with one aspect of the present invention, there is provided a method for preparing a powered mixture of plant sterol and emulsifier, comprising the steps of:
thermally melting an admixture comprising 30-70% by weight of at least one emulsifier and 70-30% by weight of plant sterol at 100-200° C., said emulsifier being selected from the group consisting of sucrose fatty acid esters, polyglycerine fatty acid esters, sorbitan fatty acid esters and polyoxylene sorbitan fatty acid esters;
rapidly cooling the molten admixture at 10° C. or lower for the solidification; and
pulverizing the solidified admixture into powders.

In accordance with another aspect of the present invention, there is provided a method for preparing a powered mixture of plant sterol and emulsifier, comprising the steps of:
thermally melting an admixture comprising 30-70% by weight of at least one emulsifier and 70-30% by weight of plant sterol at 100-200° C., said emulsifier being selected from the group consisting of sucrose fatty acid esters, polyglycerine fatty acid esters, sorbitan fatty acid esters and polyoxylene sorbitan fatty acid esters; and
spraying the molten admixture under cooling condition maintained at 10° C. or lower to give powders.

In accordance with a further aspect of the present invention, there are provide a powdered mixture, which is prepared by the above methods.

### BEST MODES FOR CARRYING OUT THE INVENTION

According to the present invention, plant sterol is admixed in a suitable ratio with at least one emulsifier and then the mixture is heated and melted as the first step.

The plant sterols are naturally occurring materials similar in structure to cholesterol. In the natural world, there are found a variety of plant sterols, of which sitosterol, campesterol, stigmasterol and sitostanol predominate over other sterols. In the present invention, the term “plant sterol” refers to all sterols and stanols found in plants, including sitosterol, campesterol, stigmasterol, sitostnaol, campestanol, etc.

Examples of the emulsifier useful in the present invention include sucrose fatty acid ester, polyglycerine fatty acid ester, sorbitan fatty acid ester, and polyoxylene sorbitan fatty acid ester.

The most important thing in admixing the plant sterol with the emulsifier is to uniformly distribute the emulsifier in plant sterol to suppress the aggregation of plant sterol, thereby obtaining high emulsion stability. In order to achieve a homogeneous admixture of the plant sterol and the emulsifier, conventionally, an organic solvent is used to dissolve both the plant sterol and the emulsifier, followed by the removal of the organic solvent. However, the admixture is not suitable for use in foods because of the possibility that a portion of the organic solvent remain in the admixture.

Instead of employing organic solvents, heat may be used to homogeneously admix plant sterol with emulsifiers. In this regard, large quantities of emulsifiers are required. However, too much emulsifier content is not suitable for use in foods. Thus, it is desirable that as small an emulsifier is used as possible, with the proviso that the plant sterol particles are prevented from aggregating together. In the present invention, an optimal admixture is obtained from 30-70% by weight of the plant sterol and 70-30% by weight of the emulsifier. For example, when plant sterol is used in an amount larger than 70% by weight, the foods to which the admixture is applied can contain much plant sterol, while the plant sterol cannot be homogeneously distributed with such relatively small amounts of the emulsifier, that poor dispersion stability occurs. On the other hand, when the plant sterol is used in an amount smaller than 30% by weight, the dispersion stability is enhanced, while the resulting food acquires the taste of the emulsifier, as well as being poor in physical properties.

In the present invention, plant sterol is homogeneously admixed with an emulsifier by heat melting. The temperature for heat-melting the plant sterol and the emulsifier is preferably on the order of 100 to 200° C. For example, when the plant sterol and emulsifier is heated at less than 100° C., they are not sufficiently admixed. On the other hand, larger than 200° C. causes the emulsifier to be denatured.

According to the present invention, the molten mixture thus obtained can be powdered in the following two manners: firstly, the molten mixture is quenched at 10° C. or lower, followed by pulverization into powder; secondly, the molten mixture is sprayed at ° C. or lower to give powder, for instance by use of nozzle.

The above two methods consider the fact that the cooling conditions of the molten admixture will have a great influence on the dispersion stability in water. That is, when dispersed in water, the powder prepared by rapidly cooling the molten admixture of plant sterol and emulsifier exhibits more excellent dispersion stability than that prepared by slowly cooling a molten admixture.

To examine why different dispersion stabilities are generated according to the cooling methods, powders obtained by rapidly and slowly cooling were analyzed with differential scanning calorimetry (DSC). As a result of DSC analysis, a plurality of peaks were observed in the DSC curve of the powdered mixture prepared through slowly cooling, whereas only one peak was detected in the DSC curve of the powdered mixture prepared through rapid cooling.

In the case of slowly cooling the molten admixture of plant sterol and emulsifier, DSC peaks are observed at each melting point of the plant sterol and the emulsifier. The reason therefor is, we believe, that the plant sterol is homogeneously mixed with the emulsifier in the molten state, and each of the ingredients constituting the admixture is deposited and aggregated in the decreasing order of the melting points as the cooling is proceeded slowly. Thus, in DSC analysis, a peak appears at each melting point. That is, in the powered admixture of plant sterol and emulsifier, which shows a plurality of peaks upon DSC analysis, the plant sterol is not homogeneously admixed with the emulsifier, but aggregates into large particles. Thus, such a powdered mixture is poor in the dispersion stability in water.

In contrast, when a molten admixture of plant sterol and emulsifier is rapidly cooled, only one DSC peak is observed at a point different from each melting point, indicating that the plant sterol and the emulsifier are admixed with each other as fine particles to homogeneity. Thus, the powdered admixture of plant sterol and emulsifier prepared by rapid cooling is stably dispersed in water.

In the present invention, the cooling condition, e.g., a cooling booth is preferably maintained at 10° C. or less and more preferably at −10° C. or less. Sufficient cooling to achieve homogeneity is not conducted at higher than 10° C.

The powdered mixture, whether it is obtained by rapidly cooling for solidification and then pulverizing a solidified admixture or by spraying the molten admixture under a cooling condition, preferably has a maximum particle size of up to 5 mm and more preferably up to 1 mm, with an average particle size ranging from 200 to 500 μm. When the maximum particle size is above 5 mm, it takes much time to conduct the emulsification, and in some cases, the particle size of the dispersed plant sterol amounts to several micrometers or larger, giving unpleasant effects on the texture of the food products.

As described above, DSC analysis can give information about the homogeneity of a powdered mixture of plant sterol and emulsifier, thus determining its dispersion stability and applicability to foods. Sucrose fatty acid ester, polyglycerine fatty acid ester, sorbitan fatty acid ester and polyoxylene sorbitan fatty acid ester are found to bring about good results in DSC analysis. Particularly, excellent is a powdered mixture which shows one DSC peak at a point of at least 100° C. Sitosterol is melted at about 140° C., campesterol at about 157° C., and stigmasterol at about 170° C. When the melting point of the powdered mixture of plant sterol and emulsifier is similar to that of the plant sterol, the plant sterol can homogeneously admixed with the emulsifier, and the powdered mixture has excellent dispersion stability in water. Such a powdered mixture may be sufficiently emulsified in hot water even by simple stirring. Generally, O/W- or W/O-type emulsions can be obtained by high-speed stirring with the aid of a homo-mixer. However, the mixture of the present invention can be emulsified by simple stirring, which results from the fact that fine particles of the plant sterol are distributed with those of the emulsifier to homogeneity.

The hydrophilic lipophilic balance (HLB) values of the emulsifier used in the powdered mixture may vary depending on the physical properties of the food base to be applied, wherein the emulsifier preferably has an HLB value of 8 or higher and more preferably 10 or higher for hydrophilic food bases such as beverage, ketchup, yogurt, etc, and preferably has an HLB value of less than 8 and more preferably less than 5 for lipophilic food bases such as mayonnaise, margarine, ice-cream, etc.

The powdered mixture of plant sterol and emulsifier prepared in accordance with the present invention can be conveniently applied to a variety of foods, irrespective of food bases, with no influence on the characteristic taste and flavor of the applied food. Also, the powdered mixture is dispersed as so fine particles in water that it can be used in a relatively small amount thanks to the high bio-availability of the plant sterol contained therein, in addition to not producing a bristly sensation in the mouth.

Having generally described this invention, a further understanding can be obtained by reference to certain specific examples which are provided herein for purposes of illustration only and are not intended to be limiting unless otherwise specified.

### COMPARATIVE EXAMPLES 1 TO 7

In a 100 mL vessel, plant sterol (sitosterol 75%, campesterol 10%, and stigrnasterol and sitostanol 15%) was admixed at the weight ratios shown in Table 1, below, with sucrose stearyl ester (HLB 11) and polyglycerine stearyl ester (HLB 12), followed by melting the mixtures at 140° C. with stirring. After being completely melted, the mixtures were slowly cooled by being allowed to stand for 10 hours at room temperature. Storage of the mixtures in a refrigerator (−10° C.) for a short period of time produced powders. The powdered mixtures of plant sterol and emulsifiers were analyzed for homogeneity by DSC and the results are given in Table 1, below.

TABLE 1

| C. Exmp. | Plant | ¹SSE | ²PSE | DSC Peak |
|---|---|---|---|---|
| No. | Sterol | (HLB 11) | (HLB 12) | (° C.) |
| 1 | 5 g | 4.25 g | 1 g | 56, 116, 125 |
| 2 | 5 g | — | 5 g | 50, 112.7, 130 |
| 3 | 5 g | — | 3.85 g | 50.1, 125.2, 135 |
| 4 | 5 g | — | 2.5 g | 49, 90.6, 130.97 |
| 5 | 5 g | 4.25 g | — | 42.96, 118.83, 131.98 |
| 6 | 5 g | 2.0 g | — | 43.25, 119.14, 132.50 |
| 7 | 5 g | 1.0 g | 1.0 g | 53.25, 112.7, 124.5 |

Note:
¹Sucrose Stearyl Ester
²Polyglycerine Stearyl Ester

### EXAMPLES 1 TO 5

In a 100 mL vessel, plant sterol (sitosterol 75%, campesterol 10%, and stigmasterol and sitostanol 15%) was admixed at the weight ratios shown in Table 2, below, with sucrose stearyl ester (HLB 11, mp. 49-55° C.) and polyglycerine stearyl ester (HLB 12, m.p. 45-55° C.), followed by melting the mixtures at 140° C. with stirring. After being completely melted, the mixtures were rapidly cooled by being stored in a refrigerator (−10° C.). The solids thus obtained were pulverized into powders with a maximum particle size of 5 mm or less. The powdered mixtures of plant sterol and emulsifiers were analyzed for homogeneity by DSC and the results are given in Table 2, below.

TABLE 2

| Exmp. | Plant | ¹SSE | ²PSE | DSC Peak |
|---|---|---|---|---|
| No. | Sterol | (HLB 11) | (HLB 12) | (° C.) |
| 1 | 5 g | 4.25 g | 1 g | 112.5 |
| 2 | 5 g | — | 5 g | 120.25 |
| 3 | 5 g | — | 3.85 g | 126 |
| 4 | 5 g | — | 2.5 g | 130.58 |
| 5 | 5 g | 4.25 g | — | 115.62 |

Note:
¹Sucrose Stearyl Ester
²Polyglycerine Stearyl Ester

As seen in Tables 1 and 2, when slowly cooled, the powdered mixtures showed DSC peaks at various temperature points, whereas only one peak was observed in the DSC curve of each of the powdered mixtures rapidly cooled, indicating that the plant sterol was homogeneously admixed with the emulsifiers.

### EXAMPLE 6

In a 100 mL vessel, 5 g of plant sterol (sitosterol 75%, campesterol 10%, and stigmasterol and sitostanol 15%) was admixed with 4.25 g of sucrose stearyl ester (HLB 11) and 1 g of polyglycerine stearyl ester (HLB 12), followed by melting the mixture at 140° C. with stirring. After being completely melted, the mixture was sprayed through a conventional nozzle for spray drying under cooling conditions at −10° C. to produce fine particles with an average size of 300 μm. DSC results of the mixture were similar to those obtained example 1.

### COMPARATIVE EXAMPLES 8 TO 14

The powdered mixtures obtained in Comparative Examples 1 to 7 were added to water maintained at 90° C., wherein the weight ratio of the powdered mixture and water was 1:100, and stirred at 800 rpm for 10 min. Their dispersibility in water is given in Table 3, below

TABLE 3

| C. Exmp. | Powdered | Dispersibility in Water |
|---|---|---|
| No. | Mix. | (After 5 days) |
| 8 | C. Exmp. 1 | Fairly unstable (settlement) |
| 9 | C. Exmp. 2 | Fairly unstable (settlement) |
| 10 | C. Exmp. 3 | Unstable (settlement) |
| 11 | C. Exmp. 4 | Very unstable (settlement) |
| 12 | C. Exmp. 5 | Fairly unstable (settlement) |
| 13 | C. Exmp. 6 | Fairly unstable (settlement) |
| 14 | C. Exmp. 7 | Fairly unstable (settlement) |

### EXAMPLES 7 TO 12

The powdered mixtures obtained in Examples 1 to 6 were added to water maintained at 90° C., wherein the weight ratio of the powdered mixture and water was 1:100, and stirred for 800 rpm for 10 min. Their dispersibility in water is given in Table 4, below

TABLE 4

| Exmp. | Powdered | Dispersibility in Water |
|---|---|---|
| No. | Mix. | (After 5 days) |
| 7 | Exmp. 1 | Very stable |
| 8 | Exmp. 2 | Very stable |
| 9 | Exmp. 3 | Fairly stable |
| 10 | Exmp. 4 | Stable |
| 11 | Exmp. 5 | Very stable |
| 12 | Exmp. 6 | Very stable |

Measurement of Disability in Water

A 1% dipersion of plant sterol in water was filled in a 100 mL mass cylinder and allowed to stand for 3 days at 25° C. To the settlement volume, the following standards were applied.

| Settlement Vol. | Judgment |
|---|---|
| 1 ml or less | Very stable |
| 3 ml or less | Fairly stable |
| 5 ml or less | Stable |
| 10 ml or less | Unstable |
| 20 ml or less | Fairly unstable |
| More than 20 ml | Very unstable |

### EXAMPLE 13

The powdered mixture prepared in Example 5 was measured for particle size and the results are given in Table 5, below. As apparent from the data of Table 5, the powdered mixture was very fine in size.

TABLE 5

| Particle Size(μm) | Cumulative % |
|---|---|
| 0.096 | 4.32 |
| 0.127 | 12.52 |
| 0.153 | 22.38 |
| 0.184 | 35.57 |
| 0.222 | 50.79 |
| 0.294 | 72.43 |
| 0.985 | 98.90 |

### Industrial Applicability

Taken together, the data obtained in the above Examples and Comparative Examples demonstrate that the powdered mixture of plant sterol and emulsifier according to the present invention can be conveniently applied to various foods irrespectively of food bases, and is excellent in terms of dispersion stability in water. Also, the powdered mixture is such a size that it is greatly improved in bio-availability, and is not bristly in the mouth, with no influence on the characteristic taste and flavor of the foods.

1. A method for preparing a powdered mixture of plant sterol and emulsifier, comprising the steps of:
thermally melting an admixture comprising 30-70% by weight of at least one emulsifier and 70-30% by weight of plant sterol at 100-200° C., said emulsifier being selected from the group consisting of sucrose fatty acid esters, polyglycerine fatty acid esters, sorbitan fatty acid esters and polyoxylene sorbitan fatty acid esters;
rapidly cooling the molten admixture at 10° C. or lower to solidify the admixture; and
pulverizing the solidified admixture into powders.

2. A method for preparing a powdered mixture of plant sterol and emulsifier, comprising the steps of:
thermally melting an admixture comprising 30-70% by weight of at least one emulsifier and 70-30% by weight of plant sterol at 100-200 8 C, said emulsifier being selected from the group consisting of sucrose fatty acid esters, polyglycerine fatty acid esters, sorbitan fatty acid esters and polyoxylene sorbitan fatty acid esters; and
spraying the molten admixture under cooling conditions maintained at 108 C or lower to give powders.

3. The method as set forth in claim 1, wherein the powdered mixture has a maximum particle size of 5 mm or less.

4. The method as set forth in claim 1, wherein the powdered mixture has a melting point of at least 1008 C as measured by a differential scanning calorimeter.

5. The method as set forth in claim 4, wherein the powdered mixture shows one melting peak on a differential scanning calorimetry plot.

6. A powdered mixture of plant sterol and emulsifier, prepared by thermally melting an admixture comprising 30-70% by weight of at least one emulsifier and 70-30% by weight of plant sterol at 100-2008 C, said emulsifier being selected from the group consisting of sucrose fatty acid esters, polyglycerine fatty acid esters, sorbitan fatty acid esters and polyoxylene sorbitan fatty acid esters; rapidly cooling the molten admixture at 108 C or lower to solidify the admixture; and pulverizing the solidified admixture into powders.

7. A powdered mixture of plant sterol and emulsifier, prepared by thermally melting an admixture comprising 30-70% by weight of at least one emulsifier and 70-30% by weight of plant sterol at 100-2008 C, said emulsifier being selected from the group consisting of sucrose fatty acid esters, polyglycerine fatty acid esters, sorbitan fatty acid esters and polyoxylene sorbitan fatty acid esters; and spraying the molten admixture under cooling conditions maintained at 108 C or lower to give powders.

8. The powdered mixture as set forth in claim 6, wherein the powdered mixture has a maximum particle size of 5 mm or less.

9. The powered mixture as set forth in claim 6, wherein the powdered mixture has a melting point of at least 1008 C as measured by a differential scanning calorimeter.

10. The powdered mixture as set forth in claim 9, wherein the powdered mixture shows one melting peak on a differential scanning calorimetry plot.

11. The method as set forth in claim 2, wherein the powdered mixture has a maximum particle size of 5 mm or less.

12. The method as set forth in claim 2, wherein the powdered mixture has a melting point of at least 100° C., as measured by a differential scanning calorimeter.

13. The method as set forth in claim 12, wherein the powdered mixture shows one melting peak on a differential scanning calorimetry plot.

14. The powdered mixture as set forth in claim 7, wherein the powdered mixture has a maximum particle size of 5 mm or less.

15. The powdered mixture as set forth in claim 7, wherein the powdered mixture has a melting point of at least 100° C., as measured by a differential scanning calorimeter.

16. The powdered mixture as set forth in claim 15, wherein the powdered mixture shows one melting peak on a differential scanning calorimetry plot.
